(19) United States
(12) Patent Application Publication
(10) Pub. No.: US 2026/0048165 A1
(43) Pub. Date: Feb. 19, 2026

(54) ORGANIC WASTE STORAGE AND DEODORIZER DEVICE

(71) Applicant: Ivan ARBOUZOV, Southlake, TX (US)

(72) Inventor: Ivan ARBOUZOV, Southlake, TX (US)

(21) Appl. No.: 19/104,507

(22) PCT Filed: Aug. 15, 2023
(86) PCT No.: PCT/US2023/030290

§ 371 Date: Feb. 18, 2025

Related U.S. Application Data
(60) Provisional application No. 63/398,098, filed on Aug. 15, 2022.

(51) Int. Cl. A61L 9/12

(52) U.S. Cl. CPC A61L 9/122; A61L 2209/111; A61L 2209/14; A61L 2209/212

(57) ABSTRACT

In general, this disclosure relates to an apparatus that reduces spoilage and the associated odors using two methods. First, air is circulated into, throughout, and out of the storage device, removing moisture and reducing the rate at which the organic matter spoils. Second, an ozone generator converts oxygen into ozone, which chemically reacts with the organic waste and odor producing chemicals within the device, reducing the discernible odor.

### CLAIM OF PRIORITY

This application claims priority to U.S. Provisional Ser. No. 63/398,098 filed on Aug. 15, 2022, the entire contents of which are hereby incorporated by reference.

### TECHNICAL FIELD

The present disclosure relates to an apparatus, system, and method for storage of organic waste prior to future processing.

### BACKGROUND

Common trash receptacles often emit a foul odor if organic waste is stored within them as moisture and the enclosed environment tends to accelerate spoiling of the organic waste.

### SUMMARY

The present disclosure involves systems, methods, and an apparatus for storing organic waste. One example implementation includes a removable bin and a cover that is configured to fit over the removable bin and includes a recirculation fan that recirculates air throughout the bin. The cover also includes an ozone generator that disperses ozone into the bin, an exhaust fan that discharges air form the bin through an exhaust port, a filter positioned in a flow path between the exhaust fan and the exhaust port, and an intake port.

Implementations can optionally include one or more of the following features.

In some instances, the storage device includes a support that the cover is affixed to, and the removable bin removable nests within the support. In some instances, the support includes a telescoping arm that the cover is affixed to, and the telescoping arm moves the cover relative to the bin.

In some instances, the intake port includes a ducted flow path through the cover, the ducted flow path requiring intake air to pass through the bin before reaching the recirculation fan or the exhaust fan.

In some instances, the exhaust fan generates a negative pressure within the bin relative to the ambient atmospheric pressure.

In some instances, the storage device includes a controller that is configured to adjust a speed of the recirculation fan, a speed of the exhaust fan, and a power level of the ozone generator.

In some instances the controller adjusts the speed of the exhaust fan based on a difference in pressure determined based on a pressure inside the bin and the ambient pressure.

In some instances, when the cover is separated from the bin, the controller is configured to stop the recirculation fan, exhaust fan, and ozone generator.

In some instances, the storage device includes a gas sensor that measures a concentration of ozone within the bin, and the controller adjusts the power level of the ozone generator based on the sensed concentration of ozone.

An example implementation includes a method for storing organic waste including containing the organic waste in a bin, positioning a cover over the bin that recirculates air throughout the bin with a first fan, discharges air from the bin via a filter using a second fan, and disperses ozone into the bin.

The details of these and other aspects and embodiments of the present disclosure are set forth in the accompanying drawings and the description below. Other features, objects, and advantages of the disclosure will be apparent from the description, drawings, and the claims.

### BRIEF DESCRIPTION OF THE DRAWINGS

FIG. 1 illustrates a front perspective view of a storage device for organic waste.

FIG. 2 illustrates a rear perspective view of a storage device for organic waste.

FIG. 3 illustrates a bottom view of a cover for a storage device for organic waste.

FIG. 4 illustrates a perspective view of a storage device with the bin removed.

FIG. 5 illustrates some of the internal components of a cover of a storage device, with certain elements removed.

FIG. 6 is a block diagram illustrating a controller and some sensors and systems the controller can actuate.

### DETAILED DESCRIPTION

In general, the disclosure relates to an apparatus, system, and method of use, for a waste storage bin that is designed to limit odor emission and waste spoilage. In general food waste or organic waste spoils relatively quickly when left in a bin or trash can. This produces an undesirable odor and can lead to increased waste. Furthermore, by combining organic waste with other waste, the total waste production of a household or organization is increased. Instead, organic waste (e.g., food waste) can be stored separately, and later reprocessed in a useful manner (e.g., in energy production, or composted, etc.). In order to allow organic waste to be stored and transported for processing, a storage device is described.

The storage device reduces spoilage and the associated odors using two methods. First, air is circulated into, throughout, and out of the storage device, removing moisture and reducing the rate at which the organic matter spoils. Second, an ozone generator converts oxygen into ozone, which chemically reacts with the organic waste and odor producing chemicals within the device, reducing the discernible odor.

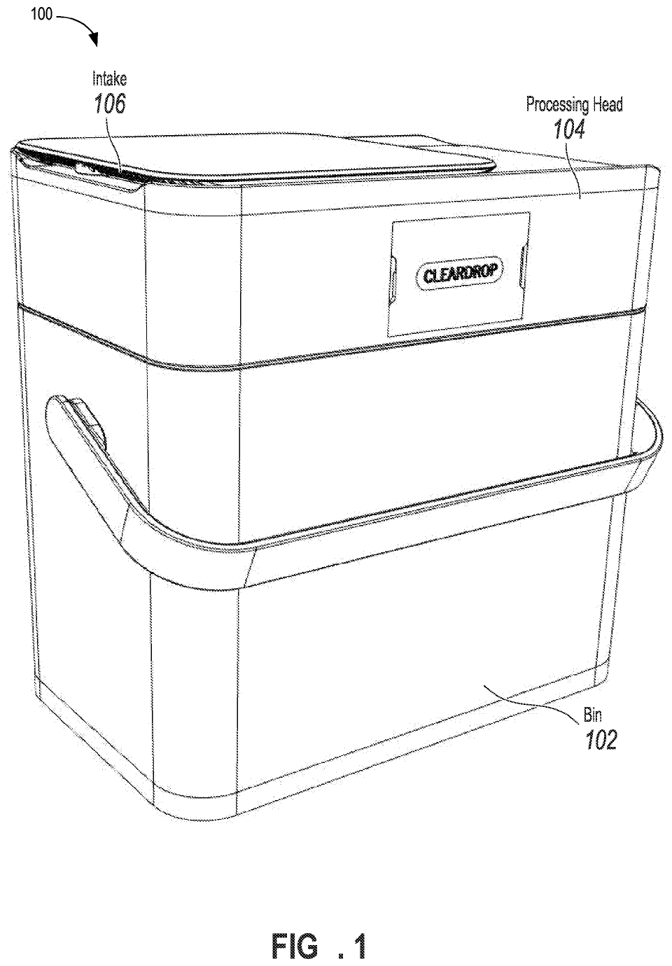

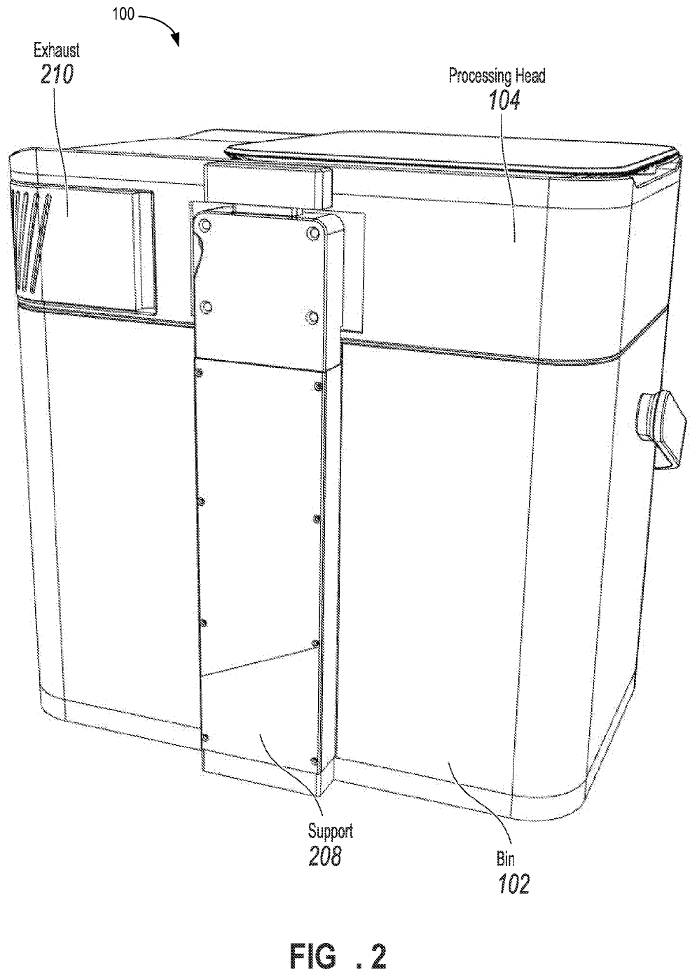

FIG. 1 illustrates a front perspective view of a storage device 100 for organic waste. Storage device 100 includes a bin 102 and a processing head 104. The bin 102 can be removable and allows for easy access to add or remove organic waste to the storage device 100. The processing head 104 includes an intake 106 through which fresh air can be drawn into the device 100. An exhaust port (not shown) can be provided to allow exhaust of air from the device, and is described in further detail below with references to FIG. 2.

Although illustrated as having a rectangular cross section, storage device 100 can take many form factors. For example, storage device 100 could be cylindrical, square, triangular, or any suitable shape. Additionally storage device 100 can be taller, or shorter than illustrated. For example, storage device 100 could be tall, such that the bin 102 is sized to fit a standard household trash bag (e.g., 13 gallons, or other volume). In some implementations, bin 102 is designed to operate without any bag, and can have an interior coating that inhibits food or other waste from adhering to it.

FIG. 2 illustrates a rear perspective view of a storage device 100 for organic waste. Illustrated in FIG. 2 is a support 208 that can retain the bin 102 and support and position the processing head/cover 104. In some implementations, the support 208 includes a telescoping arm that can raise and lower the processing head 104 in order to provide access to the bin 102. The Support 208 can include a spring loaded, electric, hydraulic, mechanical, or other mechanism for moving the processing head 104. In some implementations, the processing head 104 is translated directly upward, separating it from the bin 102. In some implementations, the processing head pivots open, rotating to provide access to the bin 102.

An exhaust 210 is provided on the processing head/cover 104 which allows airflow to leave the storage device 100. While illustrated on a rear corner of the processing head 104, exhaust 210 can be positioned in any suitable location (e.g., the top, front, or other sides). In some implementations, exhaust 210 can include a duct or a chimney, and vent gasses from the storage device 100 outside or to a location that is separate from where the storage device 100 is positioned.

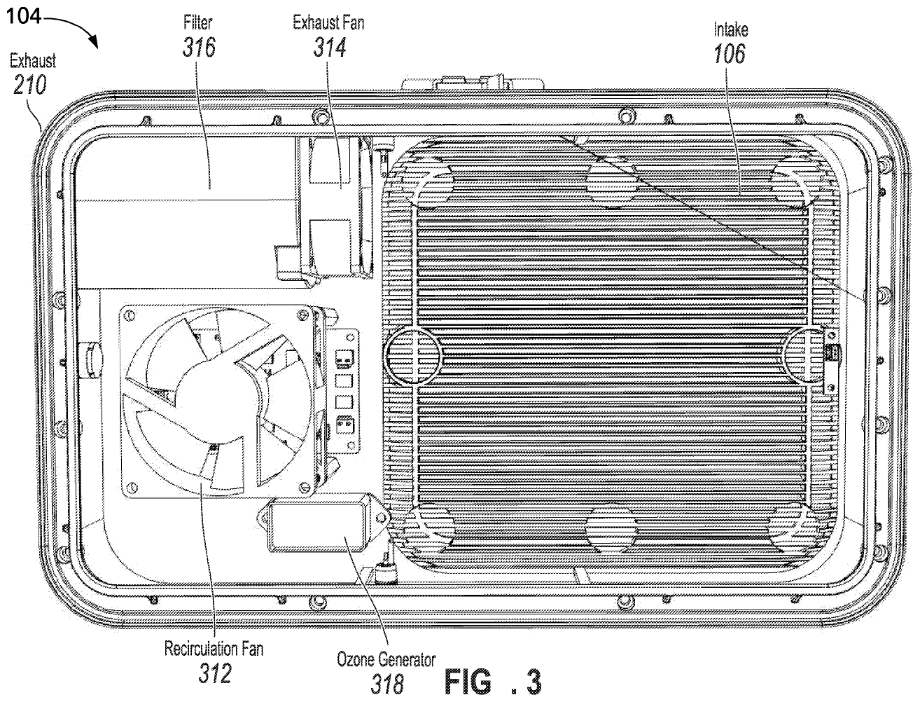

FIG. 3 illustrates a bottom view of a cover for a storage device for organic waste. The cover or processing head 104 includes a recirculation fan 312, exhaust fan 314, filter 316 and an ozone generator 318.

The recirculation fan 312 is configured to force air to mix and flow throughout the device, and generally enables rapid evaporation of any moisture that may be present in the organic waste stored in the device.

The exhaust fan 314 is configured to draw a suction on the interior region of the device, and discharge air out the exhaust 210 via filter 316. In some implementations exhaust fan 314, intake 106, and exhaust 210 are sized such that the exhaust fan generally draws a vacuum, or negative pressure with respect to ambient, on the storage device. By maintaining the bin at a pressure lower than atmospheric pressure, gas leakage is biased into the bin, instead of outward, ensuring that a majority of the gas leaving the bin passes through filter 316.

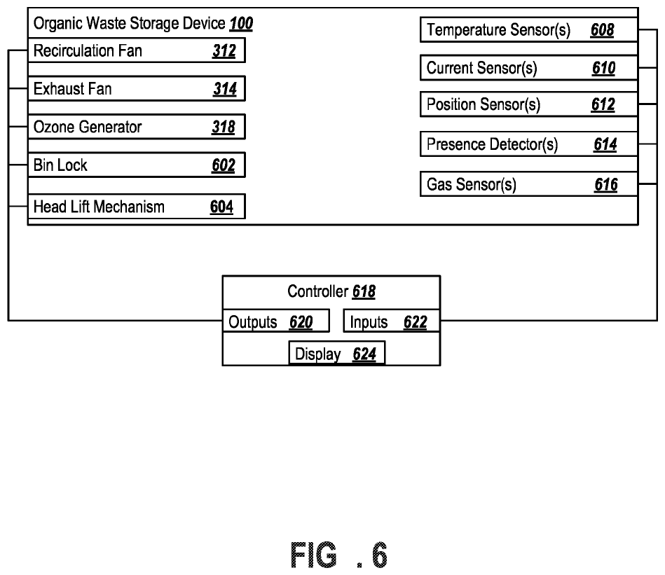

The exhaust fan 314 and the recirculation fan 312 can be the same, or different fans. In some implementations, they are brushless DC fans configured to operate at a range of different speeds, which can be operated by a controller (e.g., controller 618 as discussed below with respect to FIG. 6).

Filter 316 can be positioned in a flow path between exhaust fan 314 and the exhaust 210, and can filter any air exiting the device 100. The filter 316 can be a particulate filter such as an activated charcoal filter, or a high efficiency particulate air (HEPA) filter. In some implementations filter 316 includes an ionic removal device, or a resin exchange layer to remove ions or other charged particles from the air. In some implementations, filter 316 includes multiple layers or stages, and can have both an activated charcoal, HEPA, ionic, or other stages included. Filter 316 can be an active (e.g., energy consuming) system, or a passive system. In some implementations, filter 316 is easily removable from the processing head 104, and can be detached and replaced (e.g., after a certain time period, or number of hours of operation of the exhaust fan 314.) In some implementations, filter 316 includes one or more aromatic layers, which add a pleasing scent to the air passing through them.

Ozone generator 318 can be mounted inside the cover 104 and can convert oxygen in the storage device 100 to ozone. In some implementations, the ozone generator 318 is configured to intake air or oxygen near the top of the cover 104, and discharge ozone rich air near the bottom. Because ozone is generally heavier than air, it will tend to disperse down into the bin 102, and concentrate where the organic waste is stored (the bottom of the bin 102). There the ozone will either react with odor producing constituents of the organic waste, or decay naturally back into oxygen. The ozone generator can create ozone using a silent corona discharge reaction, where a large voltage is present across two or more electrodes, ionizing the air between them, and causing generation of ozone. In some implementations, the ozone generator uses ultraviolet (UV) light to generate ozone. Air passing into the ozone generator is bombarded with narrowband UV light causing oxygen (O₂) molecules to break apart and recombine with other oxygen molecules as ozone. In some implementations, the ozone generator is configured to activate only when conditions within the bin are suitable for ozone generation. For example, if moisture in the bin exceeds a predetermined threshold, and the ozone generator uses silent corona discharge, it can be disabled while the moisture remains above the threshold to minimize generation of nitric acid. In some implementations a moisture sensor, and gas sensor is present in the cover 104. Additionally, in certain instances, the device 100 can measure a concentration of ozone in the bin, and adjust the ozone production of the ozone generator 318 to achieve a target concentration.

In some implementations, a presence detector or switch identifies when the bin 102 has been removed from the device 100. If the bin 102 is removed, or the processing head/cover 104 is separated from the bin (e.g., the telescoping arm of support 208 is extended) than the recirculation fan 312, exhaust fan 314, and ozone generator 318 can be turned off, or de-energized.

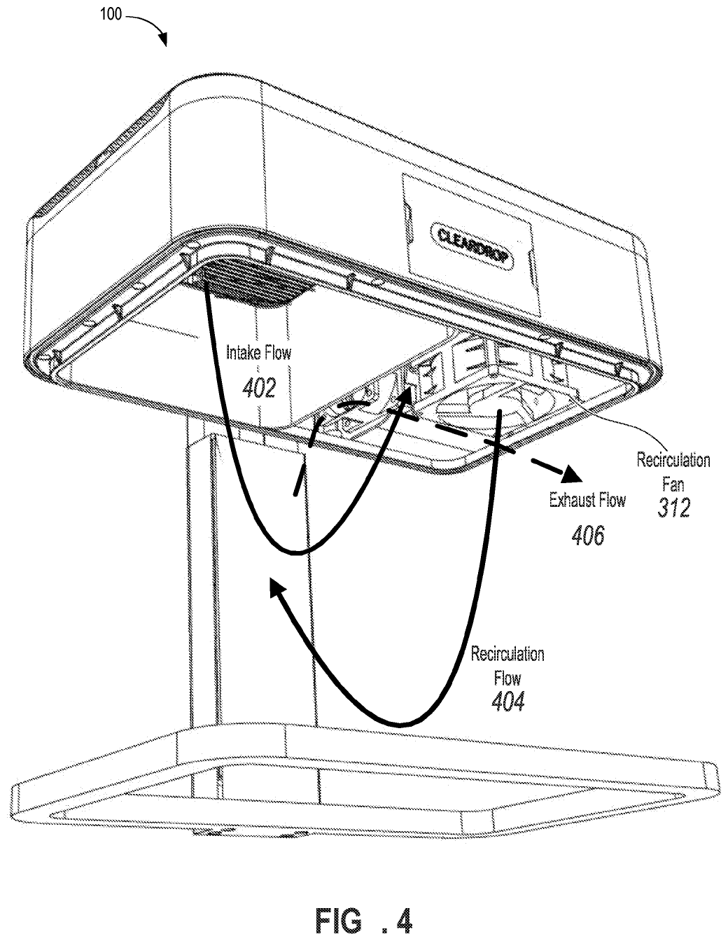

FIG. 4 illustrates a perspective view of a storage device 100 with the bin removed. The intake 106 allows an intake flow 402 to pass into the bin and mix with air circulating throughout the device 100. Visible in FIG. 4 is a duct surrounding the intake 106 which directs the intake flow 402 into the bin, instead of directly to the suction of either fan in the processing head 104 (e.g., recirculation fan 312, or exhaust fan 314). In some implementations the duct extends below the processing head 104 into the bin.

Recirculation flow 404 is produced by recirculation fan 312, which draws a suction from a region inside the cover 104 and forces mixing and circulation of air in the bin 102. In the illustrated implementation, recirculation fan 312 is positioned a stand-off distance from the top of the cover 104, to prevent high air velocity at the inlet of the recirculation fan 312.

Exhaust flow 406, illustrated with a dashed line removes air from the bin, passing it through the filter 316 prior to allowing it to leave the device 100.

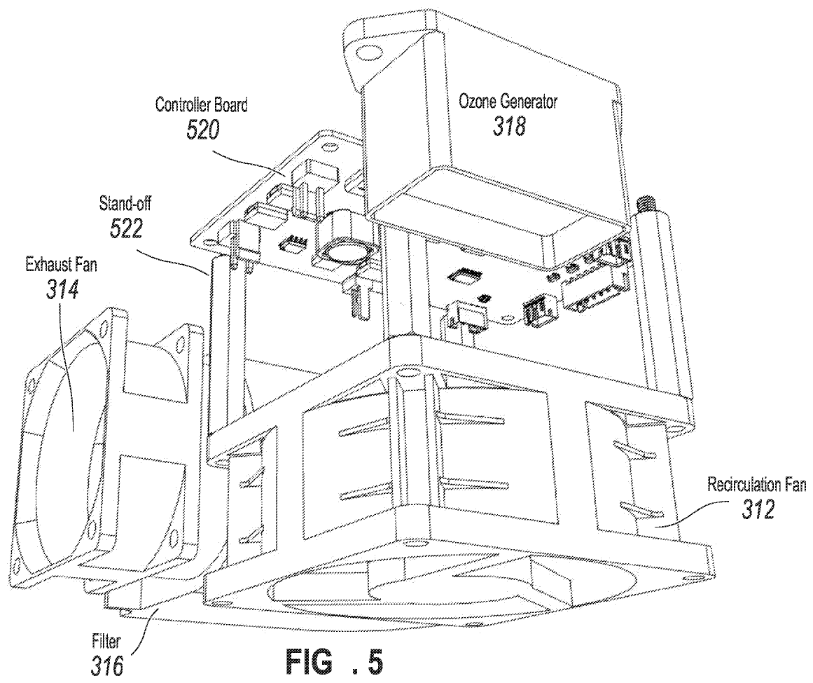

FIG. 5 illustrates some of the internal components of a cover of a storage device, with certain elements removed. Specifically many structural elements, as well as electrical components (e.g., wires, connectors, etc.) have been removed for clarity. The recirculation fan 312 can be seen mounted to a set of stand-offs 522 which provide a volume at the suction of the recirculation fan 312 to reduce air velocity at the fan's intake. Additionally a controller board 520 is provided. The controller board 520 can provide inputs to any electronic components of the storage device 100. For example the controller board 520 can command fan speed of recirculation fan 312, exhaust fan 314, as well power level of the ozone generator 318. In some implementations sensors can be mounted on, or remote from controller board 520 and provide input to the controller board 520 that can be used to modify operation of various components of the device. For example, a remote latch sensor (not illustrated) can sense whether or not the bin is installed in the device. If the bin is removed, controller board 520 can de-energize the exhaust fan 314, recirculation fan 312, and ozone generator 318.

In some implementations, the controller board 520 is a printed circuit board (PCB) which can include integrated sensors. For example an ambient pressure sensor, temperature sensor, or humidity sensor, that is affixed directly to the controller board 520. In some implementations, the controller board 520 is coated in an anti-corrosion or waterproofing material. For example controller board 520 can be coated in a conformal coating comprising polyurethane, silicon, acrylic, a combination thereof, or other coating.

FIG. 6 is a block diagram illustrating a controller 618 and some sensors and systems the controller can actuate. The organic waste storage device 100 can be communicatively coupled with a controller 618. While illustrated in FIG. 6 as separate components, the controller 618, or a portion of the controller 618, can be integrated into the organic waste storage device 100.

The controller 618 can receive inputs 622 from various sensors within the organic waste storage device 100. These inputs can include a temperature signal from one or more temperature sensors 608. The temperature sensors 608 can be thermocouples, resistance temperature detectors (RTDs), thermistors, or other suitable temperature sensors. Temperature sensors 608 can be located within a bin of the storage device, or near the processing head. Controller 618 can further receive inputs 630 from one or more current sensors 636, which can provide an indication of electrical current supplied to various components in the organic waste storage device 100 (e.g., recirculation fan 312, exhaust fan 314, ozone generator 318, etc.). One or more position sensors 612 can also provide inputs 630 to the controller. The position sensors 612 can be, for example, encoders connected to actuators associated with the cover (e.g., processing head 104 or support 208 as described with respect to FIGS. 1 and 2). In some implementations, position sensors 612 can be Hall Effect sensors, or an array of Hall Effect sensors, which sense magnetic fields and are able to determine the location of various components of the organic waste storage device 100 (e.g., processing head 104, support 208, bin 102 etc.).

One or more gas sensors 616 can provide controller 618 with information regarding the relative concentrations and pressures of gas in the organic waste storage device 100. Gas sensors 616 can include a humidity sensor, which detects a concentration of water vapor inside of and/or outside of the bin. An ozone sensor can detect a concentration of ozone within the bin. An internal pressure sensor can detect an internal pressure, and an external pressure sensor can detect an ambient atmospheric pressure. Additionally, one or more pressure sensors can be positioned at the intake of the exhaust fan 314 and the output of the filter (e.g., filter 316 of FIG. 3) in order to calculate a differential pressure across the filter. This differential pressure can be used to determine the health of the filter, or alter the user that the filter needs to be replaced.

One or more presence detectors 614 can sense the presence of waste in the organic waste storage device 100. Presence detector 614 can be, for example infrared (IR) rangefinders or ultrasonic sensors, or weight/pressure sensors which detect the presence of objects in a specific region. Presence detector 614 can determine whether a new waste has been added to or removed from the bin, and in some instances, increase or decrease fan speed or ozone generation accordingly.

The controller 618 can include a display 624 or provide signals to a display 624, which can generally provide the user information on the current status and operations of the organic waste storage device 100. The display 624 can be an LCD display, OLED display, or any other suitable display. Display 624 can provide a graphical user interface to relay information to a user, as well as receive one or more inputs (e.g., via a touchscreen and soft keys, or buttons associated with the display) from the user.

The controller 618 can provide one or more outputs 620 to the system, including but not limited to, driving currents or control signals to the recirculation fan 312, the exhaust fan 314, the ozone generator 318, a bin lock 602, or a head lift mechanism 604, which can operate processing head 104 as described in FIG. 1. Outputs 620 can be electrical signals, digital or analog, or mechanical signals and outputs (e.g., rotation of a motor or gear).

The preceding figures and accompanying description illustrate example processes and systems. However, the described system (or its software or other components) contemplates using, implementing, or executing any suitable technique for performing these and other tasks. It will be understood that these processes are for illustration purposes only and that the described or similar techniques may be performed at any appropriate time, including concurrently, individually, or in combination. In addition, many of the operations in these processes may take place simultaneously, concurrently, and/or in different orders than as shown. Moreover, the described systems and flows may use processes and/or components with or perform additional operations, fewer operations, and/or different operations, so long as the methods and systems remain appropriate.

In other words, although this disclosure has been described in terms of certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure.

What is claimed is:

1. A storage device comprising:
a removable bin; and
a cover configured to fit over the bin, the cover comprising:
a recirculation fan configured to recirculate air throughout the bin;
an ozone generator configured to disperse ozone into the bin;
an exhaust fan configured to discharge air from the bin through an exhaust port;
a filter positioned in a flow path between the exhaust fan and the exhaust port; and
an intake port.

2. The storage device of claim 1, comprising a support, wherein the cover is affixed to the support and wherein the bin removably nests within the support.

3. The storage device of claim 2, wherein the cover is affixed to a telescoping arm of the support, and wherein the telescoping arm is configured to move the cover relative to the bin.

4. The storage device of claim 1, wherein the intake port comprises a ducted flow path through the cover, and wherein the ducted flow path is configured to require intake air to pass through the bin before reaching the recirculation fan or the exhaust fan.

5. The storage device of claim 1, wherein the exhaust fan is configured to generate a negative pressure within the bin relative to an ambient atmospheric pressure.

6. The storage device of claim 1, comprising a controller configured to adjust a speed of the recirculation fan, the exhaust fan, and a power level of the ozone generator.

7. The storage device of claim 6, comprising a pressure sensor configured to measure a difference in pressure inside the bin from ambient pressure, wherein the controller is configured to adjust the speed of the exhaust fan based on the difference in pressure.

8. The storage device of claim 6, wherein the controller is configured to stop the recirculation fan, exhaust fan, and ozone generator when the cover is separated from the bin.

9. The storage device of claim 6, comprising a gas sensor configured to measure a concentration of ozone within the bin, wherein the controller is configured to adjust the power level of the ozone generator based on the measured concentration of ozone.

10. A method of storing organic waste comprising:
containing the organic waste in a bin;
positioning a cover over the bin, wherein the cover is configured to:
recirculate air throughout the bin with a first fan;
discharge air from the bin via a filter and using a second fan; and
disperse ozone into the bin.

11. The method of claim 10, comprising a support, wherein the cover is affixed to the support and wherein the bin removably nests within the support.

12. The method of claim 11, wherein the cover is affixed to a telescoping arm of the support, and wherein the telescoping arm is configured to position the cover over the bin.

13. The method of claim 10, wherein cover comprises an intake port comprising a ducted flow path through the cover, and wherein the ducted flow path is configured to require intake air to pass through the bin before reaching the first fan or the second fan.

14. The method of claim 10, wherein the second fan is configured to generate a negative pressure within the bin relative to an ambient atmospheric pressure.

15. The method of claim 10, comprising adjusting a speed of the first fan, a speed of the second fan, and an amount of ozone dispersion.

16. The method of claim 15, wherein the adjusting the speed of the second fan comprises sensing a difference in pressure inside the bin from ambient pressure and adjusting the speed of the second fan based on the difference in pressure.

17. The method of claim 15, comprising stopping the, first fan, the second fan and ozone dispersion when the cover is separated from the bin.
